(19) United States
(12) Patent Application Publication
(10) Pub. No.: US 2015/0164328 A1
(43) Pub. Date: Jun. 18, 2015

(54) ULTRASOUND DIAGNOSTIC APPARATUS

(71) Applicant: KONICA MINOLTA INC., Tokyo (JP)

(72) Inventor: Hidenori YOSHITOMI, Yokohama-shi (JP)

(73) Assignee: KONICA MINOLTA INC.

(21) Appl. No.: 14/546,858

(22) Filed: Nov. 18, 2014

(30) Foreign Application Priority Data
Dec. 16, 2013 (JP) 2013-258913

(51) Int. Cl. A61B 5/00; A61B 5/11; A61B 8/15

(52) U.S. Cl. CPC A61B 5/0035; A61B 8/15; A61B 5/1128

(57) ABSTRACT

An ultrasound diagnostic apparatus configured in a manner connectable to an ultrasound probe includes a transmission unit configured to control supply of a transmission electric signal to transmit ultrasound from the ultrasound probe to a subject, a reception unit configured to acquire a reception signal which is based on reflected ultrasound received by the ultrasound probe, an ultrasound image generation unit configured to generate tomographic image data of the subject based on the reception signal, a movement reception unit configured to acquire movement information of the subject, a dynamic schematic view data generation unit configured to generate, based on the movement information, schematic view image data illustrating a movement of the subject in a schematic view, and a display processing unit configured to generate composite image data in which the tomographic image data and the dynamic schematic view image data are compounded based on time information.

The entire disclosure of Japanese Patent Application No. 2013-258913 filed on Dec. 16, 2013 including description, claims, drawings, and abstract are incorporated herein by reference in its entirety.

### BACKGROUND OF THE INVENTION

1. Field of the Invention

The present invention relates to an ultrasound diagnostic apparatus which is used in a medical field and which can be connected to an ultrasound probe.

2. Description of the Related Art

An ultrasound diagnostic apparatus is connected to an ultrasound probe, transmits ultrasound while the ultrasound probe is applied to a surface of a subject such as an abdomen or a chest of a human body, receives reflected ultrasound reflected by the subject, and acquires an ultrasound diagnostic image. Here, a function to perform recording while displaying the ultrasound diagnostic image and a general schematic view of the subject and displaying a mark, which indicates a position of the ultrasound probe, therein in order to indicate that the acquired ultrasound diagnostic image is an image of when and where the ultrasound probe is applied on the subject has been known. A general schematic view of the subject in this case is a still image (see, for example, JP 04-166141 A and JP 2004-121488 A).

Recently, for example, in an orthopedic field, there is a case of acquiring an ultrasound diagnostic image while making a subject change a state dynamically such as case of checking muscle or a skeleton of a predetermined part while moving an arm of a patient. However, in an apparatus of the related art which displays a position of an ultrasound probe on a general schematic view which is a still image, it has been difficult to understand how an ultrasound diagnostic image changes when and what kind of operation a subject performs.

### SUMMARY OF THE INVENTION

An object of the present invention is to make it easier to recognize, when an ultrasound diagnostic image is acquired while a subject changes a state dynamically, a relationship between the acquired ultrasound diagnostic image and the dynamic change in state of the subject.

To achieve the abovementioned object, according to an aspect, an ultrasound diagnostic apparatus configured in a manner connectable to an ultrasound probe reflecting one aspect of the present invention comprises: a transmission unit configured to control supply of a transmission electric signal to transmit ultrasound from the ultrasound probe to a subject; a reception unit configured to acquire a reception signal which is based on reflected ultrasound received by the ultrasound probe; an ultrasound image generation unit configured to generate tomographic image data of the subject based on the reception signal; a movement reception unit configured to acquire movement information of the subject; a dynamic schematic view data generation unit configured to generate, based on the movement information, schematic view image data illustrating a movement of the subject in a schematic view; and a display processing unit configured to generate composite image data in which the tomographic image data and the dynamic schematic view image data are compounded based on time information.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is a view illustrating an example of a configuration of an ultrasound diagnostic apparatus in a first embodiment of the present invention;

FIG. 2 is a flowchart illustrating an example of an operation of the ultrasound diagnostic apparatus in the first embodiment of the present invention;

FIG. 3 is a view illustrating an example of movement information of a subject in the first embodiment of the present invention;

FIG. 4 is a view illustrating an example of a body mark image in the first embodiment of the present invention; and

FIG. 5A and FIG. 5B are views illustrating an example of a composite image in the first embodiment of the present invention.

### DESCRIPTION OF THE PREFERRED EMBODIMENTS

Hereinafter, an embodiment which is an example of an ultrasound diagnostic apparatus of the present invention will be described in detail with reference to the drawings. However, the scope of the invention is not limited to the illustrated examples.

### First Embodiment

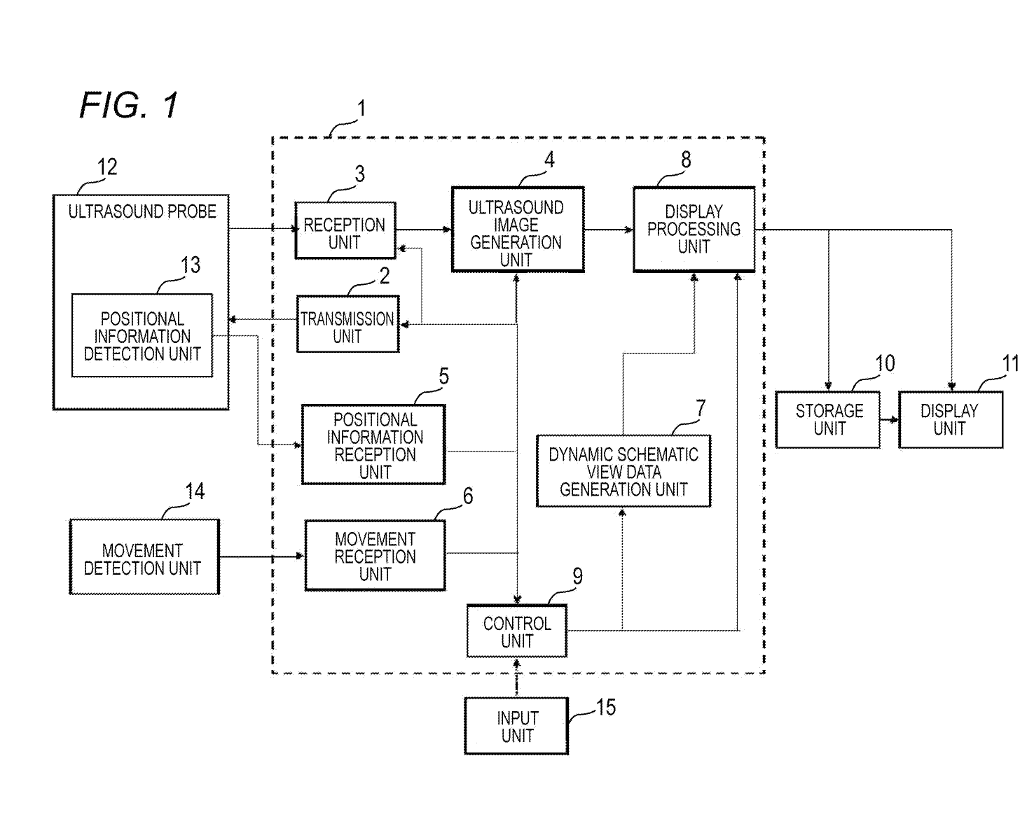

FIG. 1 is a block diagram illustrating an example of a configuration of an ultrasound diagnostic apparatus according to the first embodiment of the present invention.

The ultrasound diagnostic apparatus 1 of the present embodiment includes a transmission unit 2, a reception unit 3, an ultrasound image generation unit 4, a positional information reception unit 5, a movement reception unit 6, a dynamic schematic view data generation unit 7, a display processing unit 8, and a control unit 9. Then the ultrasound diagnostic apparatus 1 is configured in a manner connectable to a storage unit 10, a display unit 11, an ultrasound probe 12, a movement detection unit 14, and an input unit 15.

The ultrasound probe 12 includes a positional information detection unit 13 to detect positional information which is a position, an inclination, or a combination thereof of the ultrasound probe 12.

Specifically, the positional information detection unit 13 is a gyro sensor, a sensor to detect an inclination of the ultrasound probe 12, or the like.

The ultrasound probe 12 includes a vibrator to convert a transmission electric signal transmitted from the transmission unit 2 into ultrasound. The ultrasound emitted from the vibrator is transmitted to a subject while the ultrasound probe 12 is applied to a surface of the subject. Then, the ultrasound probe 12 receives reflected ultrasound reflected by the subject, converts the reflected ultrasound into a reception electric signal with the vibrator, and supplies the reception electric signal to the reception unit 3. Note that the ultrasound probe 12 may include a plurality of vibrators arrayed in one-dimensional direction.

Also, the movement detection unit 14 detects a movement of the subject to be an object of an ultrasound diagnosis. Specifically, the movement detection unit 14 is, for example, a video camera which can take a motion picture or a sensor which is attached to the subject and detects speed, acceleration, a position, or an inclination. Also, these may be combined. The transmission unit 2 which is one of the configurations included in the ultrasound diagnostic apparatus 1 generates a transmission control signal to perform transmission control of the ultrasound transmitted from the ultrasound probe 12 to the subject. Then, the transmission unit 2 performs transmission processing to supply, to the ultrasound probe 12, a transmission electric signal at high voltage which signal is generated at predetermined timing based on the transmission control signal. Note that the transmission processing performed by the transmission unit 2 at least means processing to generate the transmission control signal in the transmission unit 2 and to make the ultrasound probe 12 transmit the ultrasound. The transmission unit 2 includes a pulsar, a transmission beam former, and the like.

The reception unit 3 receives a reception electric signal from the ultrasound probe 12, performs processing, which is necessary for construction or the like of ultrasound tomographic image data, such as amplification or detection of the reception electric signal, and performs reception processing to generate a reception signal. Note that the reception processing performed by the reception unit 3 at least means processing in which the reception unit 3 acquires the reception signal which is based on the reflected ultrasound. For example, in a case where the plurality of vibrators is arrayed in the one-dimensional direction, the reception unit 3 generates the reception signal by performing amplification and A/D conversion of the reception electric signal converted by the ultrasound probe 12. Then, by giving appropriate delay to the reflected ultrasound received by each of the vibrators and by performing adding, only ultrasound from a predetermined position or direction is detected. By performing the transmission processing by the transmission unit 2 and the reception processing by the reception unit 3, a plurality of reception signals corresponding to one image frame is acquired. By repeating these pieces of processing for a plurality of times, a plurality of reception signals corresponding to a plurality of image frames is acquired. The reception unit 3 includes an amplifier, an A/D converter, a reception beam former, and the like.

Note that a part of the functions of the transmission unit 2 and the reception unit 3 may be provided on a side of the ultrasound probe 12. For example, the following configuration can be considered. That is, in the ultrasound probe 12, a transmission electric signal is generated based on a transmission control signal which is output from the transmission unit 2 and which is for generating a transmission electric signal. Then, the transmission electric signal is converted into ultrasound and received reflected ultrasound is converted into a reception electric signal, and then, a reception signal is generated in the ultrasound probe 12 based on the reception electric signal and the reception unit 3 receives the reception signal.

Generally, the transmission unit 2 performs the transmission processing repeatedly and continuously and generates a reception signal successively. Thus, the following processing is performed successively relative to the generated reception signal.

The ultrasound image generation unit 4 receives the reception signal generated in the reception unit 3, analyzes amplitude of the reception signal, converts the reception signal into a luminance signal corresponding to signal intensity thereof, performs coordinate transformation or the like of the luminance signal in such a manner that the luminance signal corresponds to an orthogonal coordinate system, and successively constructs tomographic image data (B mode image data) which is an ultrasound image. The ultrasound image generation unit 4 includes, for example, various filters, a detector, a logarithmic amplifier, a scan converter, a different signal/image processor, and the like. Note that since the reception signal is digitalized, the conversion of the reception signal into a luminance signal corresponding to signal intensity of the reception signal may be realized not by the above-described hardware but by software.

The positional information reception unit 5 is connected to the positional information detection unit 13 included in the ultrasound probe 12 and receives positional information of the ultrasound probe 12 which information is detected by the positional information detection unit 13. The movement reception unit 6 is connected to the movement detection unit 14 and receives information of a movement of the subject which movement is detected by the movement detection unit 14.

Based on the information of the movement of the subject which information is received by the movement reception unit 6, the dynamic schematic view data generation unit 7 generates schematic view image data illustrating the movement of the subject in a schematic view. Moreover, based on the positional information of the ultrasound probe 12 which information is received by the positional information reception unit 5, the dynamic schematic view data generation unit 7 generates, in the schematic view image data illustrating the movement of the subject in a schematic view, body mark image data which simultaneously illustrates a position or an inclination of the ultrasound probe 12.

The display processing unit 8 generates composite image data in which the tomographic image data generated by the ultrasound image generation unit 4, the schematic view image data or the body mark image data generated by the dynamic schematic view data generation unit 7, and the like are compounded. Then, the display processing unit 8 performs processing to store the composite image data into the storage unit 10 or to display the composite image data onto the display unit 11.

The control unit 9 includes, for example, an arithmetic processor including a memory and controls an operation of each configuration.

The storage unit 10 is an electronic storage medium such as a memory and stores the composite image data generated by the display processing unit 8. The storage unit 10 may be embedded in the ultrasound diagnostic apparatus 1.

The display unit 11 is, for example, a display and displays the composite image data generated by the display processing unit 8. Also, the composite image data stored in the storage unit 10 may be displayed. The display unit 11 may be included integrally as one chassis in the ultrasound diagnostic apparatus 1.

The input unit 15 is a keyboard, a mouse, a trackball, or the like. The input unit 15 receives an input from an operator of the ultrasound diagnostic apparatus 1 and inputs, into the control unit 9, an instruction which is based on the input from the operator.

Typically, a part or the whole of a function of each function block included in the ultrasound diagnostic apparatus 1 can be realized as an LSI which is an integrated circuit. Note that here, the LSI is used but may be referred to as an IC, a system LSI, a super LSI, or an ultra LSI depending on a degree of integration. Also, a method of circuit integration is not limited to the LSI and may be realized by a special circuit or a general processor. A field programmable gate array (FPGA) or a reconfigurable processor in which connection or setting of a circuit cell inside an LSI can be reconfigured may be used. Also, a part of the whole of a function of each function block may be executed by software. In this case, the software is stored in one or more storage media, optical disks, or hard disks. The storage media are, for example, ROMs. The software is executed by the arithmetic processor.

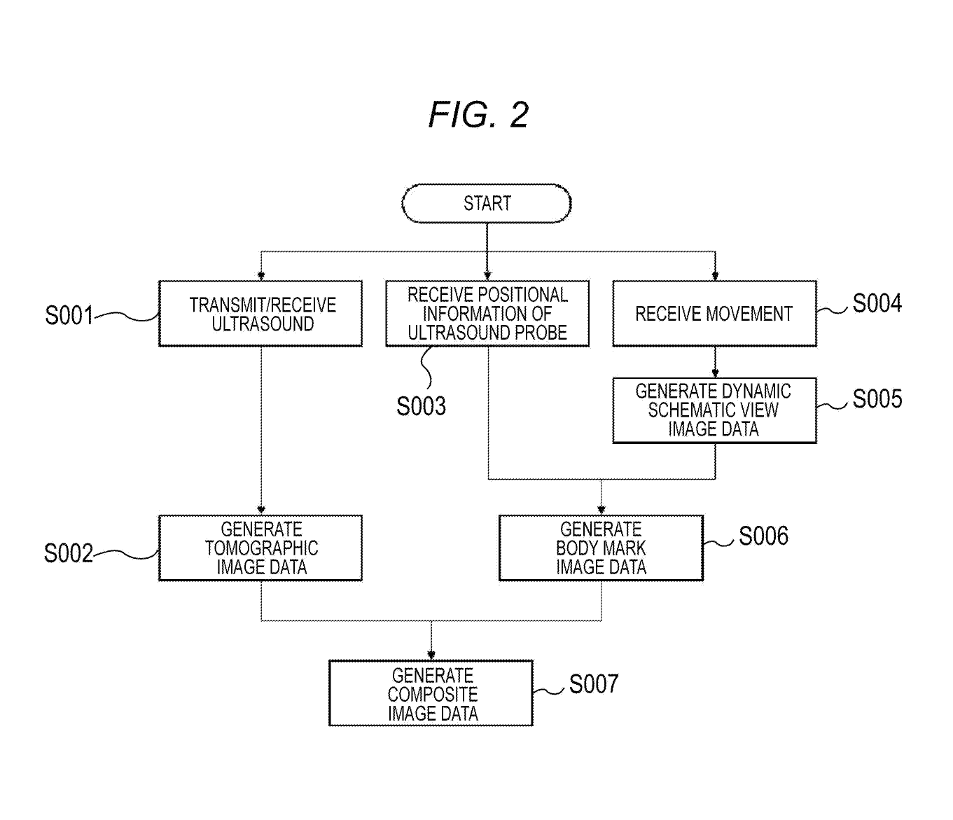

Next, an operation of the ultrasound diagnostic apparatus 1 including such a configuration described above will be described with reference to FIG. 2. FIG. 2 is a flowchart illustrating an example of the operation of the ultrasound diagnostic apparatus 1 in the first embodiment of the present invention.

First, the ultrasound probe 12 is arranged on a surface of the subject and the ultrasound diagnostic apparatus 1 transmits/receives ultrasound (step S001). Specifically, an example in which the ultrasound probe 12 is arranged on a shoulder of a human body will be described. The ultrasound diagnostic apparatus 1 successively acquires a reception signal including a cross-sectional surface of a muscle or a bone of the shoulder by the transmission processing and the reception processing respectively by the transmission unit 2 and the reception unit 3 and acquires a plurality of reception signals corresponding to one image frame. Moreover, the acquisition is repeated and reception signals corresponding to a plurality of image frames in a predetermined period of time are acquired.

Next, the ultrasound image generation unit 4 receives the plurality of reception signals acquired in step S001, analyzes amplitude of the reception signals, converts each of the reception signals into a luminance signal corresponding to signal intensity thereof, performs coordinate transformation or the like of the luminance signals in such a manner that the luminance signals correspond to an orthogonal coordinate system, and successively constructs tomographic image data including a cross-sectional surface of a muscle or a bone of the shoulder. Here, time at which acquisition of a plurality of reception signals corresponding to one image frame is completed is recorded into a memory or the like as time information associated with the one image frame (step S002).

On the other hand, the positional information reception unit 5 acquires, from the positional information detection unit 13, positional information which is a position, an inclination, or a combination thereof of the ultrasound probe 12. Here, time at which the positional information is acquired is recorded into the memory or the like as time information associated with the positional information (step S003).

Moreover, by a video camera which is the movement detection unit 14, a certain range including the shoulder of the human body which is the subject is shot. The movement reception unit 6 acquires movement information of a movement of the human body including the shoulder which is the subject shot by the video camera. Here, a period of time in which the movement is performed is recorded into the memory or the like as time information associated with the movement information (step S004).

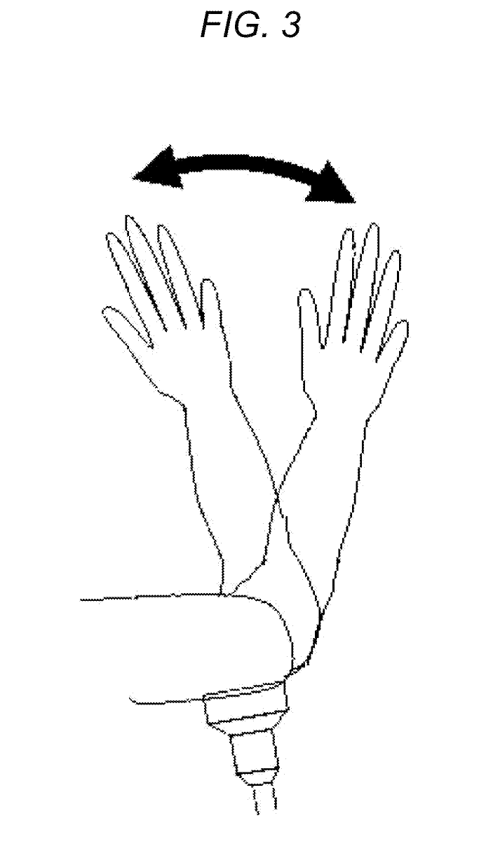

Next, based on the movement information acquired in step S004, the dynamic schematic view data generation unit 7 generates schematic view image data which illustrates, in a schematic view, the movement of the human body including the shoulder of a person who is the subject (step S005). Specifically, motion picture information acquired from the video camera is received by the movement reception unit 6 and the dynamic schematic view data generation unit 7 compares changes in color and/or luminance value data of an image between the frames, analyzes the changes of the image between the frames, and extracts an outline of a dynamic image. Then, image data of a background and that of the other static part or image data of a part in which a movement is equal to or less than a predetermined value is deleted and motion picture data which is outline data of a dynamic image extracted from the motion picture information is generated as dynamic schematic view data. FIG. 3 is a view illustrating an example of the motion picture of when the ultrasound probe 12 is applied to the shoulder and a movement to move a right arm to a left side (inward rotation) and a movement to move the right arm to a right side (outward rotation) are performed with an elbow as a supporting point. A state in which a person who is the subject is seen from a head side is illustrated. In an image shot by the video camera, a background or the like is shot along with the human body. From the motion picture, luminance value data of the image is analyzed and an outline of the moving arm and that of the shoulder and the human body connected thereto are extracted as a schematic view of the human body.

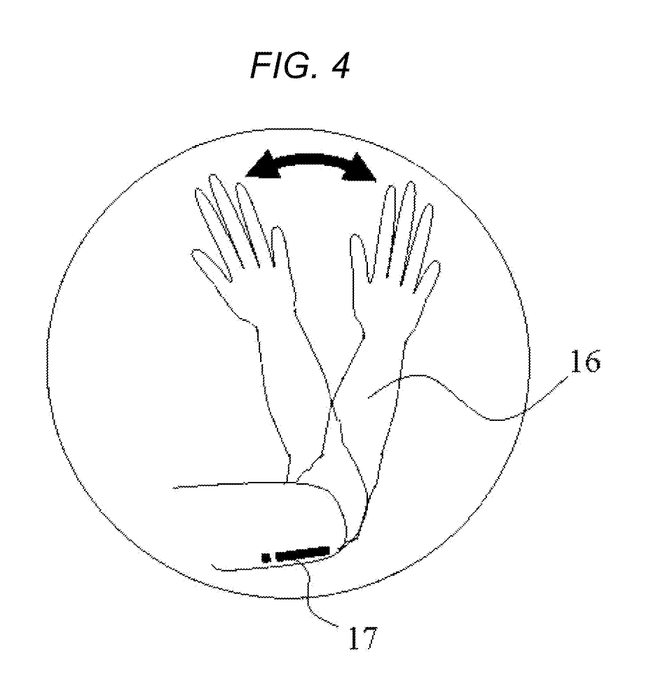

Here, the display processing unit 8 displays, on the display unit 11, the dynamic schematic view data generated in step S005. Here, the image to be displayed may be displayed as a screen in which the dynamic schematic view data and the tomographic image data generated in step S002 are compounded. Then, by using the input unit 15, an operator of the ultrasound diagnostic apparatus 1 specifies, in the dynamic schematic view data, a part on the screen which part corresponds to a region to which the ultrasound probe 12 is applied. The display processing unit 8 receives an input of the specified part, generates body mark image data which displays a schematic view of the ultrasound probe 12 on the specified part, and displays the body mark image data onto the display unit 11 (step S006). As a method to specify, in the dynamic schematic view data, a region to which the ultrasound probe 12 is applied, an outline of the ultrasound probe 12 may be extracted from color and/or luminance value data of the image in each frame of the motion picture information shot by the video camera and a position to which the ultrasound probe 12 is applied may be automatically superimposed on the dynamic schematic view data. Here, by comparison with data of color and/or shape of the ultrasound probe 12 recorded into the memory or the like in advance, the ultrasound probe 12 is detected from the image. Moreover, the positional information reception unit 5 receives angle information of an inclination detection sensor from the positional information detection unit 13 in the ultrasound probe 12 The display processing unit 8 generates data based on the angle information in such a manner that an angle of the schematic view of the ultrasound probe 12 in the body mark image data becomes identical to an angle of the ultrasound probe 12 actually applied to the subject. Also, based on positional information of the ultrasound probe 12 which information is acquired at time identical to the time information associated with the movement information of the motion picture of the dynamic schematic view data, a position or an angle of the schematic view of the ultrasound probe 12 changes along with a change in time of the dynamic schematic view data. FIG. 4 is a view illustrating an example of displaying the body mark image data on the display unit 11. A schematic view of the human body 16 is a display based on the dynamic schematic view data and is a motion picture. The view repeats inward rotation and outward rotation with the lapse of time. When a position or an angle in an ultrasound probe schematic view 17 displayed on the schematic view of the human body 16 changes with the lapse of time, the position or the angle changes on the schematic view of the human body 16.

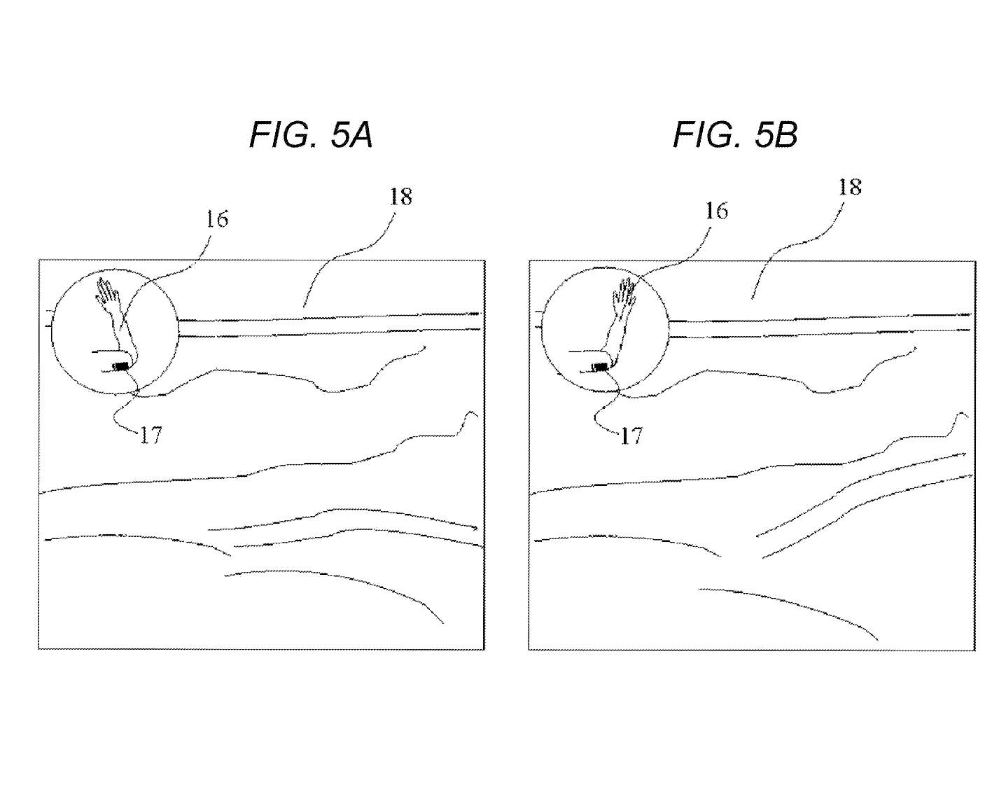

Next, the display processing unit 8 generates composite image data in which the tomographic image data generated in step S002 and the body mark image data generated in step S006 are compounded (step S007). Here, body mark image data which is based on dynamic schematic view data and positional information of the ultrasound probe 12 which are acquired at the time identical to time information associated with one image frame of the tomographic image data is compounded. FIG. 5A and FIG. 5B are views illustrating an example of displaying the composite image data on the display unit 11. FIG. 5A is a view of when the right arm is rotated inward and FIG. 5B is a view of when the right arm is rotated outward. On the display unit 11, an image in which a tomographic image 18 and the body mark image are compounded is displayed.

Then, the ultrasound diagnostic apparatus 1 displays the composite image data generated in step S007 on the display unit 11 and/or stores the data into the storage unit 10. The control unit 9 may read the data stored in the storage unit 10 and may display the read data on the display unit 11. According to such a configuration, the following can be visually recognized as a motion picture. That is, an ultrasound diagnostic image of which position of the subject is what kind of image when the subject is in what kind of movement state.

### Modification of First Embodiment

In the first embodiment, in step S005, schematic view image data is generated by analyzing luminance value data of an image and extracting an outline of a dynamic image in motion picture information acquired by the video camera. However, a plurality of patterns of schematic view data of a motion picture of a subject may be stored in a memory and a pattern may be selected by an operator on an input unit 15 or a pattern similar to a generated dynamic schematic view may be selected automatically. Here, for example, a sensor to detect speed, acceleration, a position, or an inclination may be attached to an arm, a fingertip, or the like of the subject as a movement detection unit 14 and a movement reception unit 6 may detect a movement of the arm in respect to a lapse of time, and thus, a dynamic schematic view data generation unit 7 may generate dynamic schematic view data in which a movement speed or an angle of a motion picture pattern selected by the operator is made identical to a movement speed or an angle of the actual arm. That is, in step S004, acquisition of movement information of a movement of a human body is performed not by the video camera but by the sensor to detect speed, acceleration, a position, or an inclination. Other operations and configurations of an ultrasound diagnostic apparatus 1 are similar to those of the first embodiment, and thus, description thereof is omitted.

Application to an ultrasound diagnostic apparatus or the like, which is used when a dynamic change of an ultrasound diagnostic image is observed with an observed region being moved, is possible.

According to an embodiment of the present invention, since a movement reception unit configured to acquire movement information of a subject, a dynamic schematic view data generation unit configured to generate schematic view image data illustrating a movement of the subject in a schematic view based on the movement information, and a display processing unit configured to generate composite image data in which tomographic image data and the dynamic schematic view image data are compounded based on time information are included, the ultrasound diagnostic image can be visually recognized along with the movement of the subject.

Although the present invention has been described and illustrated in detail, it is clearly understood that the same is by way of illustrated and example only and is not to be taken by way of limitation, the scope of the present invention being interpreted by terms of the appended claims.

What is claimed is:

1. An ultrasound diagnostic apparatus configured in a manner connectable to an ultrasound probe, comprising:
a transmission unit configured to control supply of a transmission electric signal to transmit ultrasound from the ultrasound probe to a subject;
a reception unit configured to acquire a reception signal which is based on reflected ultrasound received by the ultrasound probe;
an ultrasound image generation unit configured to generate tomographic image data of the subject based on the reception signal;
a movement reception unit configured to acquire movement information of the subject;
a dynamic schematic view data generation unit configured to generate, based on the movement information, schematic view image data illustrating a movement of the subject in a schematic view; and
a display processing unit configured to generate composite image data in which the tomographic image data and the dynamic schematic view image data are compounded based on time information.

2. The ultrasound diagnostic apparatus according to claim 1, wherein the dynamic schematic view data generation unit extracts an outline of the subject and generates the schematic view image data based on the movement information.

3. The ultrasound diagnostic apparatus according to claim 2, wherein the dynamic schematic view data generation unit generates the schematic view image data based on predetermined schematic view data stored in advance and the movement information.

4. The ultrasound diagnostic apparatus according to claim 1, further comprising a positional information reception unit configured to receive positional information of the ultrasound probe,
wherein the dynamic schematic view data generation unit generates, based on the positional information, body mark image data in which the schematic view image data and the positional information of the ultrasound probe are compounded.
